(19) United States
(12) Patent Application Publication
(10) Pub. No.: US 2020/0234830 A1
(43) Pub. Date: Jul. 23, 2020

(54) METHOD AND DATA PROCESSING UNIT FOR SELECTING A RISK ASSESSMENT COMPUTER PROGRAM

(71) Applicant: Siemens Healthcare GmbH, Erlangen (DE)

(72) Inventors: Maria Jimena COSTA, Nuernberg (DE); Klaus HEISSNER, Erlangen (DE); Kirstin JATTKE, Nuernberg (DE); Ali KAMEN, Skillman, NJ (US)

(73) Assignee: Siemens Healthcare GmbH, Erlangen (DE)

(21) Appl. No.: 16/744,761

(22) Filed: Jan. 16, 2020

(30) Foreign Application Priority Data
Jan. 23, 2019 (EP) 19153273.8

(51) Int. Cl. G16H 50/30; G16H 10/60; G16H 50/20

(52) U.S. Cl. CPC G16H 50/30; G16H 50/20; G16H 10/60

(57) ABSTRACT

A computer-implemented method is for displaying a graphical user interface containing a selection element to select one risk assessment computer program out of a patient-related subset of a plurality of risk assessment computer programs of a patient on a display unit for an user. In an embodiment, the method includes retrieving a set of disease-related workflows; retrieving a plurality of risk assessment computer programs; selecting a disease-related dataset from the patient-related data record; determining at least one of the disease-related workflow stages from the set of disease-related workflows based on the selected disease-related dataset; determining a patient-related subset of the plurality of risk assessment computer programs based on the determined at least one of the disease-related workflow stage; and displaying the graphical user interface.

### PRIORITY STATEMENT

The present application hereby claims priority under 35 U.S.C. § 119 to European patent application number EP19153273.8 filed Jan. 23, 2019, the entire contents of which are hereby incorporated herein by reference.

### FIELD

The invention generally relates, in one embodiment, to a computer-implemented method for displaying a graphical user interface that contains a selection element to select one risk assessment computer program out of a patient-related subset of a plurality of risk assessment computer programs of a patient on a display unit for an user. In another embodiment, the invention generally relates to a data processing unit, a computer program product and a computer-readable medium.

### BACKGROUND

In the decision-making process within a hospital environment typically risk assessment computer programs are used by a physician to decide on the management of a patient. Such a risk assessment computer program can be an essential tool for the physician because it usually provides an accurate estimate of risk. Risk assessment computer programs can be used for designing a clinical trial, e.g., to ensure that homogeneous and/or high-risk patient groups are included. One advantage of the risk assessment computer programs can be the increased performance in predicting probabilities of clinical outcome of the managed patient compared to clinical judgment of the physician. The risk assessment computer programs are typically used at different points in the patient's evolution and/or clinical pathway, for instance, early to predict cancer on initial biopsy and/or on repeat biopsy, or at a later time point to predict biochemical recurrence after specific treatment of the cancer.

Usually over 100 different risk assessment computer programs are available to the physician and/or provided within a computer network, e.g. the Internet. However, the proper selection of the risk assessment computer program suitable at that specific point in the patient's evolution and/or clinical pathway can be very challenging for the physician.

### SUMMARY

An underlying technical problem of at least one embodiment of the invention provides a computer-implemented method for displaying a graphical user interface that contains a selection element to select one risk assessment computer program out of a patient-related subset of a plurality of risk assessment computer programs of a patient on a display unit for an user, a data processing unit, a computer program product and a computer-readable medium with increased usability for the physician.

This problem may solved by the features of the at least one embodiment. Advantageous embodiments are disclosed within the claims.

The invention relates in one embodiment to a computer-implemented method for displaying a graphical user interface that contains a selection element to select one risk assessment computer program out of a patient-related subset of a plurality of risk assessment computer programs of a patient on a display unit for an user, the method comprising:
retrieving a set of disease-related workflows, each disease-related workflow of the set of disease related workflows including at least two disease-related stages and being stored within a computer network,
retrieving a plurality of risk assessment computer programs, each risk assessment computer program predicting a probability of clinical outcome and being stored within the computer network,
retrieving a patient-related data record of the patient from the computer network,
selecting a disease-related dataset from the patient-related data record,
determining at least one of the disease-related workflow stages from the set of disease-related workflows based on a first disease-related mapping function, the selected disease-related dataset being an input of the first disease-related mapping function,
determining a patient-related subset of the plurality of risk assessment computer programs based on a second disease-related mapping function, the determined at least one of the disease-related workflow stage being an input of the second disease-related mapping function, and
displaying the graphical user interface that contains the selection element to select the one risk assessment computer program out of the patient-related subset of the plurality of risk assessment computer programs of the patient on the display unit for the user.

One embodiment of the invention relates in one aspect to a method, wherein a calculable subset of the plurality of risk assessment computer programs is determined, wherein the calculable subset is used as input for determining the patient-related subset and wherein the determining the calculable subset comprises following steps:
providing a set of input parameter categories, wherein the plurality of risk assessment computer programs depends on the set of input parameter categories, each risk assessment computer program predicting the probability of clinical outcome as the function of values in the input parameter categories,
selecting a patient-related dataset from the patient-related data record, wherein the patient-related dataset comprises at least one input parameter category,
comparing the patient-related dataset with the provided set of input parameter categories of the plurality of risk assessment computer programs, whereby the calculable subset of the plurality of risk assessment computer programs is determined. This embodiment can be particularly advantageous since in addition to the at least one of the disease-related workflow stages the calculability of the plurality of risk assessment computer programs is considered.

At least one embodiment is directed to a computer-implemented method for displaying a graphical user interface containing a selection element to select one risk assessment computer program out of a patient-related subset of a plurality of risk assessment computer programs of a patient on a display unit for an user, the method comprising:
retrieving a set of disease-related workflows, each disease-related workflow of the set of disease-related workflows including at least two disease-related stages and being stored within a computer network;
retrieving a plurality of risk assessment computer programs, each risk assessment computer program, of the plurality of risk assessment computer programs, predicting a probability of clinical outcome and being stored within the computer network;
retrieving a patient-related data record of the patient from the computer network;
selecting a disease-related dataset from the patient-related data record;
determining at least one of the disease-related workflow stages from the set of disease-related workflows based on a first disease-related mapping function, the disease-related dataset selected being an input of the first disease-related mapping function;
determining a patient-related subset of the plurality of risk assessment computer programs based on a second disease-related mapping function, the at least one of the disease-related workflow stage determined being an input of the second disease-related mapping function; and
displaying the graphical user interface containing a selection element to select the one risk assessment computer program, out of the patient-related subset of the plurality of risk assessment computer programs of the patient, on the display unit for the user.

At least one embodiment is directed to a data processing unit, comprising:
a processor,
a network interface, and
the display unit, wherein the data processing is configured to perform
   retrieving a set of disease-related workflows, each disease-related workflow of the set of disease-related workflows including at least two disease-related stages and being stored within a computer network,
   retrieving a plurality of risk assessment computer programs, each risk assessment computer program, of the plurality of risk assessment computer programs, predicting a probability of clinical outcome and being stored within the computer network,
   retrieving a patient-related data record of the patient from the computer network,
   selecting a disease-related dataset from the patient-related data record,
   determining at least one of the disease-related workflow stages from the set of disease-related workflows based on a first disease-related mapping function, the disease-related dataset selected being an input of the first disease-related mapping function,
   determining a patient-related subset of the plurality of risk assessment computer programs based on a second disease-related mapping function, the at least one of the disease-related workflow stage determined being an input of the second disease-related mapping function, and
   displaying the graphical user interface containing a selection element to select the one risk assessment computer program, out of the patient-related subset of the plurality of risk assessment computer programs of the patient, on the display unit for the user.

At least one embodiment is directed to a non-transitory computer program product storing a computer program, the computer program being loadable into a memory unit of a data processing system and including program code sections to enable the data processing system to execute the method of an embodiment when the computer program is executed in the data processing system.

At least one embodiment is directed to a non-transitory computer-readable medium, storing program code sections of a computer program, the program code sections being at least one of loadable into and executable in a data processing system to enable the data processing system to execute the method of an embodiment when the program code sections are executed in the data processing system.

### BRIEF DESCRIPTION OF THE DRAWINGS

The invention will be illustrated below with reference to the accompanying figures using example embodiments. The illustration in the figures is schematic and highly simplified and not necessarily to scale.

FIG. 1 shows a diagram illustrating a method according to one embodiment of the invention.

FIG. 2 shows a diagram illustrating a method according to another embodiment of the invention.

FIG. 3 shows a diagram illustrating a method according to a further embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The invention relates in one embodiment to a computer-implemented method for displaying a graphical user interface that contains a selection element to select one risk assessment computer program out of a patient-related subset of a plurality of risk assessment computer programs of a patient on a display unit for an user, the method comprising:
retrieving a set of disease-related workflows, each disease-related workflow comprising at least two disease-related stages and being stored within a computer network,
retrieving a plurality of risk assessment computer programs, each risk assessment computer program predicting a probability of clinical outcome and being stored within the computer network,
retrieving a patient-related data record of the patient from the computer network,
selecting a disease-related dataset from the patient-related data record,
determining at least one of the disease-related workflow stages from the set of disease-related workflows based on a first disease-related mapping function, the selected disease-related dataset being an input of the first disease-related mapping function,
determining a patient-related subset of the plurality of risk assessment computer programs based on a second disease-related mapping function, the determined at least one of the disease-related workflow stage being an input of the second disease-related mapping function, and
displaying the graphical user interface that contains the selection element to select the one risk assessment computer program out of the patient-related subset of the plurality of risk assessment computer programs of the patient on the display unit for the user.

The method of at least one embodiment my in particular advantageous for at least one of following reasons:

Displaying the patient-related subset of the plurality of risk assessment computer programs reduce effort and/or time for the user to select one appropriate risk assessment computer program. Thereby typically also costs are reduced. Alternatively, or additionally a management of the patient can be improved because the displaying the graphical user interface is standardized. Another advantage can be an increased repeatability and/or reproducibility at managing the patient. The patient-related subset of the plurality of risk assessment computer programs comprises preferably such risk assessment computer programs which are relevant for the patient and/or for the particular point in the patient's evolution and/or clinical pathway.

One additional advantage of the method can be that the patient can be assigned to specific stages of disease-related workflows, so a decision-making process could be sped up. The method can be beneficial because it provides a possibility to handle a wide range (N>100) of different risk assessment computer programs easily by the user. The user can be a physician and/or a health professional.

The computer network comprises typically at least one server computer and one client computer. Usually the computer network comprises a memory unit for storing data and/or signals. The server computer can be part of a hospital network that is accessible via the Internet or is disconnected from the Internet. The server computer can be a virtual machine within another host server computer. The server computer can be physically located within a data center wherein the data center is connected to the hospital network and/or to the Internet. The client computer is typically connected to the hospital network and/or to the Internet.

Usually, the client computer and the server computer have a network interface for communicating the signals and/or the data. The communicating can comprise sending and/or retrieving the signals and/or data. The retrieving the set of disease-related workflows, the plurality of risk assessment computer programs and/or the patient-related data record occurs typically by using or via the network interface. The signals and/or data are typically sent from the server computer and retrieved by the client computer or vice versa. The retrieving can comprise a downloading and/or an uploading and/or a storing of the signals and/or the data. The data can comprise the risk assessment computer programs.

Basically, the client computer and the server computer can be physically installed within a single host server computer. In that case, the client computer and the server computer are part of the single host server computer. The client computer, the server computer and/or the host server computer comprises typically a RAM unit, a memory unit, a data processing unit and/or the network interface. The client computer can comprise the display unit. The client computer can be a stationary computer and/or a portable computer such as a smartphone and/or tablet computer. The network interface comprises usually an encryption module for encrypting the signals and/or data.

The predicted probability of clinical outcome of each risk assessment computer program correlates typically to a risk for the patient. The risk for the patient can comprise a risk for initial occurrence, a risk of recurrence and/or a risk of death. Basically, a lots of different risk types can be assessed for different types of diseases. For example, Shariat et al. describe already a set of risk assessment computer programs in the specific field of prostate cancer (“An Updated Catalog of Prostate Cancer Predictive Tools”, DOI 10.1002/cncr.23908, the entire contents of which is hereby incorporated herein by reference). The predicted probability of clinical outcome can be used to establish a diagnosis and/or prognosis for the patient. The plurality of risk assessment computer programs may comprise less than 100 computer programs, about 100 computer programs and/or more than 100 computer programs.

The risk assessment computer programs are in general an array of risk assessment formulas for various stages of treatment and/or diagnosis and/or various clinical questions. Each risk assessment computer program can be typically assigned to at least one of the following types:
nomogram,
risk grouping,
probability table,
classification tree,
regression tree,
artificial network.

The set of disease-related workflows comprises typically more than one disease-related workflow. Each disease-related workflow describes typically at least one clinical pathway of the following:
cancer,
cardiac disease,
lung disease,
anatomical disease,
blood disease.

The disease-related workflows are typically established based on technical literature and/or clinical experience by the user. Every disease-related workflow describes typically a clinical pathway and/or a patient's evolution with regard to a particular disease. The patient suffers usually from that particular disease. In general the at least two disease-related stages are part of such clinical pathway and/or patient's evolution with regard to the particular disease. The at least two disease-related stages are usually discrete stages. The at least two disease-related stages can be typically uniquely assigned to the disease-related workflows. The clinical pathway can comprise different temporal and/or local, in particular subsequent, steps of treating and/or diagnosing the patient. The patient's evolution usually comprises different phases of a progression of the disease from the patient's perspective. Such phases can be definable from low risk early stage to advance post treatment recurrence. The phases are typically discrete. The disease-related workflows are usually part of disease-related workflow computer programs and/or part of disease-related workflow rule sets that can in particular be stored within and/or retrieved from the computer network.

The patient-related data record of the patient can be stored within a picture archiving and communication system and/or a radiology information system and/or a patient information system, the systems typically being part of and/or connected to the computer network. The patient-related data record comprises usually the clinical and/or medical history and/or protected health information of the patient. The protected health information can comprise social security number, first name, last name and/or birth date of the patient.

Usually the patient-related data record can be classified into a disease-related dataset and into other data. The other data can comprise the protected health information. The disease-related dataset comprises in particular the clinical and/or medical history and/or the age of the patient. The clinical and/or medical history comprises in particular at least one of the following:
diagnostic findings,
diagnostic images,
therapeutic results,
genomics,
comorbidities,
demographic data.

The selecting the disease-related dataset may comprise the classifying. The selecting is usually conducted automatically based on a predefined rule set. The predefined rule set can be defined by the user and/or the technical literature. Basically, the selecting can be conducted by the user, for instance using the display unit. Typically, only the disease-related dataset is selected from the patient-related data record.

The determining by a disease-related mapping function typically has an effect similar to filtering, ranking and/or classifying.

The determining the at least one of the disease-related workflow stages occurs typically automatically after selecting the disease-related dataset. The at least one of the disease-related workflow stages from the set of disease-related workflows is typically the output having applied the first disease-related mapping function on the selected disease-related dataset. By determining the at least one of the disease-related workflow stages typically the disease-related dataset is linked to the set of disease-related workflows, in particular to the at least two disease-related stages. The linking can comprise a linking probability representing usually how likely the patient is at the at least one of the disease-related workflow stages. Usually only one disease-related workflow stage is determined. Alternatively, or additionally a group of disease-related workflow stages can be determined by the first disease-related mapping function wherein the group typically comprises similar and/or adjacent disease-related workflow stages.

The determining the patient-related subset of the plurality of risk assessment computer programs is conceptually similar to the preceding act of determining the at least one of the disease-related workflow stages, basically differentiating in the input and output. Usually the patient-related subset comprises more than one of the plurality of risk assessment computer programs.

The graphical user interface is typically displayed on the display unit for the user. The display unit can comprise a screen, a monitor and/or an input device such as keyboard, mouse, and/or voice control. By displaying the graphical user interface containing the selection element to select one risk assessment computer program out of a patient-related subset of the plurality of risk assessment computer programs of the patient, the selection element is typically provided for the user to select the risk assessment computer program, especially by looking at the display unit and/or using the input device.

The selection element can comprise a selection list that contains the patient-related subset of the plurality of risk assessment computer programs. The selection list is preferably sortable by the user according to relevance with respect to urgency and/or efficacy and/or precision of the patient-related subset of the plurality of risk assessment computer programs. The selection element can comprise a checkbox next to and/or a toggle button of the item being part of the patient-related subset of the plurality of risk assessment computer programs. The display unit preferably comprises a speech recognition system configured to select the item by voice control if the user wants to select the one risk assessment computer program out of the patient-related subset of the plurality of risk assessment computer programs of the patient on the display unit for the user. Alternatively, and or additionally the displaying the graphical user interface comprises displaying the non-patient-related subset of the plurality of risk assessment computer programs wherein the non-patient-related subset is highlighted differently compared to the patient-related subset on the display unit.

The risk assessment computer programs are usually provided as packed or compiled computer programs, e.g. using 7z-, zip-, exe- and/or msi-packaging. The risk assessment computer programs are typically executable on the server computer and/or the client computer. If executed on the server computer, for instance in the virtual machine, the network interface is usually configured to stream the execution of the executed risk assessment computer program to the client computer. Alternatively, or additionally the risk assessment computer programs can be executed directly on the client computer. The client computer, the host server computer and/or the server computer typically comprise at least one of the following operating systems: Windows, Linux, Unix, Android, iOS, macOS, etc. Typically, the risk assessment computer programs are adapted to the appropriate operating system.

The selected one risk assessment computer program out of the patient-related subset of the plurality of risk assessment computer programs of the patient can be executed before, during and/or after displaying the graphical user interface that contains the selection element to select the one risk assessment computer program out of the patient-related subset of the plurality of risk assessment computer programs of the patient on the display unit for the user. Advantageously can be the execution before and/or during the displaying since the user can access or apply the selected risk assessment computer program typically quicker compared to delayed execution. In other words, the user typically uses or applies the selected risk assessment computer program after the displaying of the graphical user interface. Having selected the one risk assessment computer program the user and/or another user can use or apply it for managing the patient, in particular for assessing the predicted probability of clinical outcome.

One embodiment of the invention relates in one aspect to a method, wherein a calculable subset of the plurality of risk assessment computer programs is determined, wherein the calculable subset is used as input for determining the patient-related subset and wherein the determining the calculable subset comprises following steps:
providing a set of input parameter categories, wherein the plurality of risk assessment computer programs depends on the set of input parameter categories, each risk assessment computer program predicting the probability of clinical outcome as the function of values in the input parameter categories,
selecting a patient-related dataset from the patient-related data record, wherein the patient-related dataset comprises at least one input parameter category,
comparing the patient-related dataset with the provided set of input parameter categories of the plurality of risk assessment computer programs, whereby the calculable subset of the plurality of risk assessment computer programs is determined. This embodiment can be particularly advantageous since in addition to the at least one of the disease-related workflow stages the calculability of the plurality of risk assessment computer programs is considered.

Thereby typically the time and/or effort for the user is further reduced.

The input parameter categories are at least one type of the following:
blood test,
initial biopsy,
molecular test,
age.

The set of input parameter categories can be provided by retrieving the plurality of risk assessment computer programs. The set of input parameter categories are typically defined by identifying input values of the plurality of risk assessment computer programs. In other words, the set of input parameter categories can be a set of program variables. Typically, every risk assessment computer program depends at least on one input parameter category or program variable. Usually, the patient-related data record comprises at least one input parameter categories of the set of input parameter categories and/or its corresponding result or value. Typically, the patient-related data record can be clustered or classified according to the set of input parameter categories. Usually, the clinical and/or medical history, in particular the selected disease-related dataset, comprises at least one input parameter category, especially a result or a value of such at least one input parameter category. Typically, another patient-related dataset without any input parameter category cannot be selected.

The comparing the patient-related dataset with the provided set of input parameter categories typically comprises identifying such risk assessment computer programs that are calculable using the selected disease-related dataset or using the patient-related data record. The input based on the patient-related dataset for the determining the at least one of the disease-related workflow stages can differ from the input based on the patient-related dataset for comparing the patient-related dataset with the provided set of input parameter categories. The comparing comprises basically matching the input parameter categories of the patient-related data record, in particular the selected disease-related dataset, with the input parameter categories of the plurality of risk assessment computer programs and assigning such risk assessment computer programs to the calculable subset that can be calculated using the input parameter categories of the patient-related data record, in particular the selected disease-related dataset. In other words, the calculable subset comprises typically those risk assessment computer programs that can be calculated without additional tests on or of the patient. A risk assessment computer program is usually calculable, if an output on basis of the required input parameter categories can be generated upon execution.

One embodiment of the invention relates in one aspect to a method, wherein the patient-related subset of the plurality of risk assessment computer programs is differentiated by the determined calculable subset and wherein the displaying the graphical user interface comprises displaying the differentiated patient-related subset of the plurality of risk assessment computer programs according to their calculability on the display unit for the user. One advantage of this embodiment can be the visual depiction of the calculability of the respective risk assessment computer program which usually reduces the time and/effort for assessing this information. The patient-related subset can be particularly split into a calculable subset and a non-calculable subset of risk assessment computer programs.

One embodiment of the invention relates in one aspect to a method, wherein the displaying the graphical user interface comprises identifying such input parameter categories of the patient-related subset of the plurality of risk assessment computer programs, missing for the calculability, and displaying the identified missing input parameter categories on the display unit for the user. This embodiment is particularly beneficial for the user since by considering the missing input parameter categories the patient can be correspondingly examined. Typically, at such examination the result and/or value of such missing input parameter category can be determined and added to the patient-related data record. In that case, usually the formerly non-calculable risk assessment computer program can be assigned to the calculable subset if the input parameter categories match primarily.

One embodiment of the invention relates in one aspect to a method, wherein the patient-related subset of the plurality of risk assessment computer programs is merged with the determined calculable subset, wherein a calculable patient-related subset of the plurality of risk assessment computer programs is determined, and wherein the graphical user interface is configured such that it contains the selection element to select the one risk assessment computer program out of the calculable patient-related subset of the plurality of risk assessment computer programs of the patient on the display unit for the user. An advantage can be that typically only such risk assessment computer programs are selected and/or executed that are also calculable. The merging comprises typically an AND-operation.

One embodiment of the invention relates in one aspect to a method, wherein the first disease-related mapping function is determined by training a machine learning system based on a first set of training data and/or wherein the second disease-related mapping function is determined by training the machine learning system based on a second set of training data. The training of the first disease-related mapping function and/or the second disease-related mapping function using the machine learning system is beneficial because such machine learning system can be specifically tailored to patients.

One embodiment of the invention relates in one aspect to a method, wherein the machine learning system and/or the first disease-related mapping function and/or the second disease-related mapping function is based on an artificial neural network. The artificial neural network can be advantageously executed within the computer network and/or the client computer and/or the host server computer and/or the server computer.

One embodiment of the invention relates in one aspect to a method, wherein the artificial neural network comprises a convolutional neural network. The convolutional neural network is typically very adaptable and/or efficient. The convolution neural network comprises in particular an input layer, an output layer, and an intermediate layer, the layers connected via weighted links. The training of such convolutional neural network typically comprises determining the weights of the links.

The data processing unit can be realized as a data processing system or as a part of a data processing system. The data processing system can, for example, comprise cloud-computing system, a computer network, a computer, a tablet computer, a smartphone or the like. The cloud-computing system is typically connected via the Internet. The data processing system can comprise hardware and/or software. The hardware can be, for example, a processor system, a memory system and combinations thereof. The hardware can be configurable by the software and/or be operable by the software.

The computer program product can be, for example, a computer program or comprise another element apart from the computer program. This other element can be hardware, for example a memory device, on which the computer program is stored, a hardware key for using the computer program and the like, and/or software, for example a documentation or a software key for using the computer program.

Reference is made to the fact that the described methods and the described data processing unit as well as the described imaging device are merely preferred example embodiments of the invention and that the invention can be varied by a person skilled in the art, without departing from the scope of the invention provided it is specified by the claims. The features, advantages or alternative embodiments of the apparatus apply also to the method and vice-versa.

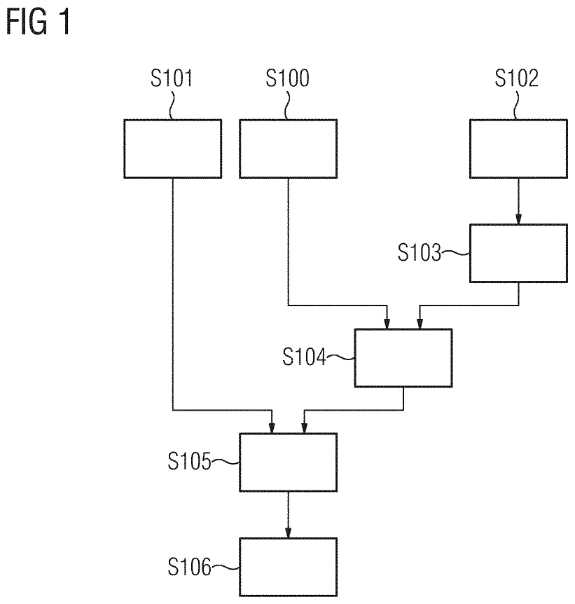

FIG. 1 shows a diagram illustrating a computer-implemented method for displaying a graphical user interface that contains a selection element to select one risk assessment computer program out of a patient-related subset of a plurality of risk assessment computer programs of a patient on a display unit for an user.

Method step S100 includes retrieving a set of disease-related workflows, each disease-related workflow comprising at least two disease-related stages and being stored within a computer network.

Method step S101 includes retrieving a plurality of risk assessment computer programs, each risk assessment computer program predicting a probability of clinical outcome and being stored within the computer network.

Method step S102 includes retrieving a patient-related data record of the patient from the computer network.

Method step S103 includes selecting a disease-related dataset from the patient-related data record.

Method step S104 includes determining at least one of the disease-related workflow stages from the set of disease-related workflows based on a first disease-related mapping function, the selected disease-related dataset being an input of the first disease-related mapping function.

Method step S105 includes determining a patient-related subset of the plurality of risk assessment computer programs based on a second disease-related mapping function, the determined at least one of the disease-related workflow stage being an input of the second disease-related mapping function.

Method step S106 includes displaying the graphical user interface that contains the selection element to select the one risk assessment computer program out of the patient-related subset of the plurality of risk assessment computer programs of the patient on the display unit for the user.

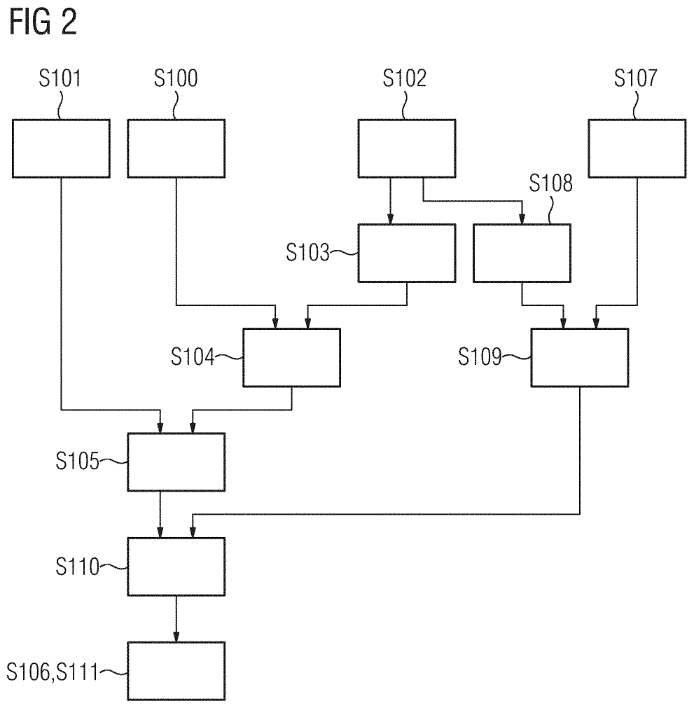

FIG. 2 shows a diagram illustrating a method according to another embodiment of the invention.

This embodiment comprises method steps S100 to S106 as shown in FIG. 1. Additionally, a calculable subset of the plurality of risk assessment computer programs is determined, wherein the calculable subset is used as input for determining the patient-related subset and wherein the determining the calculable subset comprises following steps

Method step S107 includes providing a set of input parameter categories, wherein the plurality of risk assessment computer programs depends on the set of input parameter categories, each risk assessment computer program predicting the probability of clinical outcome as the function of values in the input parameter categories.

Method step S108 includes selecting a patient-related dataset from the patient-related data record, wherein the patient-related dataset comprises at least one input parameter category.

Method step S109 includes comparing the patient-related dataset with the provided set of input parameter categories of the plurality of risk assessment computer programs, whereby the calculable subset of the plurality of risk assessment computer programs is determined.

Method step S110 includes that the patient-related subset of the plurality of risk assessment computer programs is differentiated by the determined calculable subset and wherein the displaying the graphical user interface comprises displaying the differentiated patient-related subset of the plurality of risk assessment computer programs according to their calculability on the display unit for the user.

Method step S111 includes that the displaying the graphical user interface comprises identifying such input parameter categories of the patient-related subset of the plurality of risk assessment computer programs, missing for the calculability, and displaying the identified missing input parameter categories on the display unit for the user.

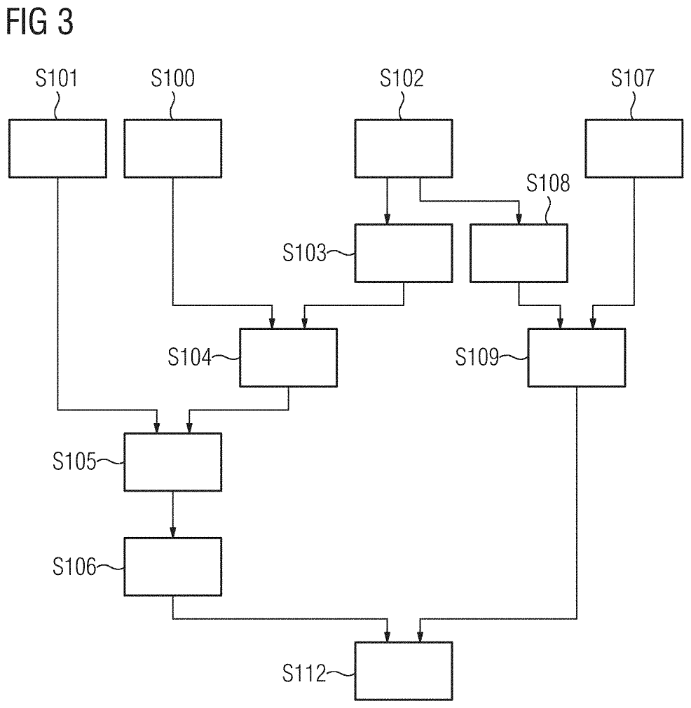

FIG. 3 shows a diagram illustrating a method according to another embodiment of the invention.

This embodiment comprises method steps S100 to S109 as shown in FIG. 1 and FIG. 2.

The method step S112 includes that the patient-related subset of the plurality of risk assessment computer programs is merged with the determined calculable subset, wherein a calculable patient-related subset of the plurality of risk assessment computer programs is determined, and wherein the graphical user interface is configured such that it contains the selection element to select the one risk assessment computer program out of the calculable patient-related subset of the plurality of risk assessment computer programs of the patient on the display unit for the user.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for displaying a graphical user interface containing a selection element to select one risk assessment computer program out of a patient-related subset of a plurality of risk assessment computer programs of a patient on a display unit for an user, the method comprising:
retrieving a set of disease-related workflows, each disease-related workflow of the set of disease-related workflows including at least two disease-related stages and being stored within a computer network;
retrieving a plurality of risk assessment computer programs, each risk assessment computer program, of the plurality of risk assessment computer programs, predicting a probability of clinical outcome and being stored within the computer network;
retrieving a patient-related data record of the patient from the computer network;
selecting a disease-related dataset from the patient-related data record;
determining at least one of the disease-related workflow stages from the set of disease-related workflows based on a first disease-related mapping function, the disease-related dataset selected being an input of the first disease-related mapping function;
determining a patient-related subset of the plurality of risk assessment computer programs based on a second disease-related mapping function, the at least one of the disease-related workflow stage determined being an input of the second disease-related mapping function; and
displaying the graphical user interface containing a selection element to select the one risk assessment computer program, out of the patient-related subset of the plurality of risk assessment computer programs of the patient, on the display unit for the user.

2. The method of claim 1, wherein a calculable subset of the plurality of risk assessment computer programs is determined, wherein the calculable subset determined is used as input for determining the patient-related subset and wherein the determining of the calculable subset comprises:
providing a set of input parameter categories, wherein the plurality of risk assessment computer programs depends on the set of input parameter categories provided, each risk assessment computer program predicting a probability of clinical outcome as a function of values in the input parameter categories,
selecting a patient-related dataset from the patient-related data record, wherein the patient-related dataset selected includes at least one input parameter category, and
comparing the patient-related dataset selected with the set of input parameter categories of the plurality of risk assessment computer programs provided, whereby the calculable subset of the plurality of risk assessment computer programs is determined.

3. The method of claim 2, wherein the patient-related subset of the plurality of risk assessment computer programs is differentiated by the calculable subset determined and wherein the displaying the graphical user interface comprises displaying the patient-related subset of the plurality of risk assessment computer programs differentiated, according to respective calculability on the display unit for the user.

4. The method of claim 3, wherein the displaying of the graphical user interface includes identifying input parameter categories of the patient-related subset of the plurality of risk assessment computer programs, missing for the calculability, and displaying the identified missing input parameter categories on the display unit for the user.

5. The method of claim 2, wherein the patient-related subset of the plurality of risk assessment computer programs is merged with the calculable subset determined, wherein a calculable patient-related subset of the plurality of risk assessment computer programs is determined, and wherein the graphical user interface is configured to contain the selection element to select the one risk assessment computer program out of the calculable patient-related subset of the plurality of risk assessment computer programs of the patient on the display unit for the user.

6. The method of claim 1, wherein at least one of
the first disease-related mapping function is determined by training a machine learning system based on a first set of training data, and
the second disease-related mapping function is determined by training the machine learning system based on a second set of training data.

7. The method of claim 6, wherein at least one of the machine learning system, the first disease-related mapping function and the second disease-related mapping function is based on an artificial neural network.

8. The method of claim 7, wherein the artificial neural network includes a convolutional neural network.

9. A data processing unit, comprising:
a processor,
a network interface, and
a display unit, wherein the data processing is configured to perform
retrieving a set of disease-related workflows, each disease-related workflow of the set of disease-related workflows including at least two disease-related stages and being stored within a computer network,
retrieving a plurality of risk assessment computer programs, each risk assessment computer program, of the plurality of risk assessment computer programs, predicting a probability of clinical outcome and being stored within the computer network,
retrieving a patient-related data record of the patient from the computer network,
selecting a disease-related dataset from the patient-related data record,
determining at least one of the disease-related workflow stages from the set of disease-related workflows based on a first disease-related mapping function, the disease-related dataset selected being an input of the first disease-related mapping function,
determining a patient-related subset of the plurality of risk assessment computer programs based on a second disease-related mapping function, the at least one of the disease-related workflow stage determined being an input of the second disease-related mapping function, and
displaying a graphical user interface containing a selection element to select the one risk assessment computer program, out of the patient-related subset of the plurality of risk assessment computer programs of the patient, on the display unit for a user.

10. A non-transitory computer program product storing a computer program, the computer program being loadable into a memory unit of a data processing system and including program code sections to enable the data processing system to execute the method of claim 1 when the computer program is executed in the data processing system.

11. A non-transitory computer-readable medium, storing program code sections of a computer program, the program code sections being at least one of loadable into and executable in a data processing system to enable the data processing system to execute the method of claim 1 when the program code sections are executed in the data processing system.

12. The method of claim 2, wherein at least one of
the first disease-related mapping function is determined by training a machine learning system based on a first set of training data, and
the second disease-related mapping function is determined by training the machine learning system based on a second set of training data.

13. The method of claim 12, wherein at least one of the machine learning system, the first disease-related mapping function and the second disease-related mapping function is based on an artificial neural network.

14. The method of claim 13, wherein the artificial neural network includes a convolutional neural network.

15. The method of claim 3, wherein at least one of
the first disease-related mapping function is determined by training a machine learning system based on a first set of training data, and
the second disease-related mapping function is determined by training the machine learning system based on a second set of training data.

16. The method of claim 15, wherein at least one of the machine learning system, the first disease-related mapping function and the second disease-related mapping function is based on an artificial neural network.

17. The method of claim 16, wherein the artificial neural network includes a convolutional neural network.

18. A non-transitory computer program product storing a computer program, the computer program being loadable into a memory unit of a data processing system and including program code sections to enable the data processing system to execute the method of claim 2 when the computer program is executed in the data processing system.

19. A non-transitory computer-readable medium, storing program code sections of a computer program, the program code sections being at least one of loadable into and executable in a data processing system to enable the data processing system to execute the method of claim 2 when the program code sections are executed in the data processing system.
